(19) United States
(12) Patent Application Publication
(10) Pub. No.: US 2005/0149103 A1
(43) Pub. Date: Jul. 7, 2005

(54) ICA angioplasty with cerebral protection

(76) Inventor: John J. "Buddy" Connors III, Tampa, FL (US)

Correspondence Address: GORE ENTERPRISE HOLDINGS, INC., 551 PAPER MILL ROAD, P. O. BOX 9206, NEWARK, DE, 19714-9206, US

(21) Appl. No.: 11/034,082

(22) Filed: Jan. 12, 2005

Related U.S. Application Data
(62) Division of application No. 10/103,309, filed on Mar. 19, 2002.
Application No. 10/103,309 is a continuation-in-part of application No. 09/835,017, filed on Apr. 13, 2001, now abandoned.
Application No. 09/835,017 is a continuation of application No. 09/018,365, filed on Feb. 4, 1998, now Pat. No. 6,295,989.
(60) Provisional application No. 60/038,040, filed on Feb. 6, 1997.
(60) Provisional application No. 60/037,226, filed on Feb. 6, 1997.
(60) Provisional application No. 60/037,225, filed on Feb. 6, 1997.
(60) Provisional application No. 60/038,039, filed on Feb. 6, 1997.

(51) Int. Cl. A61M 29/00

(52) U.S. Cl. 606/194

(57) ABSTRACT

The present invention provides a method and device for preventing cerebral embolization during endovascular procedures in the carotid arteries. The invention comprises a guide catheter and balloon, which may selectively occlude the common carotid artery, and further comprises a wire and balloon, which may selectively occlude the internal carotid artery. Occlusion of the common carotid artery will induce retrograde flow at the site, redirecting emboli to the external carotid artery. Occlusion of the internal carotid artery reduces the risk of emboli migrating to the brain, and allows clearance of the site by antegrade blood flow from the common carotid artery. Occlusion of both the common and internal carotid arteries induces a quiescent state at the site during the procedure. Control of blood flow by selective inflation and deflation of either or both balloons, in concert with the procedures, will reduce the risk of emboli migrating to the brain.

### REFERENCE TO RELATED APPLICATIONS

The present application is a divisional of U.S. patent application Ser. No. 10/103,309, filed Mar. 19, 2002, which is a continuation-in-part of U.S. patent application Ser. No. 09/835,017 filed Apr. 13, 2001, which is a continuation of U.S. patent application Ser. No. 09/018,365 filed Feb. 4, 1998, now U.S. Pat. No. 6,295,989, which claims benefit from the filing date of provisional U.S. patent application Ser. Nos. 60/038,040, 60/037,226, 60/027,225, and 60/038,039, all filed Feb. 6, 1997.

### FIELD OF THE INVENTION

This invention relates to a device and method for performing angioplasty and stenting of the internal carotid artery (“ICA”), while protecting the cerebrum from emboli dislodged or formed during the procedure. The method and device either temporarily occludes blood flow to the brain from the ICA or temporarily induces retrograde flow from the ICA. This selective control of blood flow during an interventional procedure prevents emboli from migrating to the brain during treatment of stenosis.

### BACKGROUND OF THE INVENTION

Current treatments of vascular diseases, such as stenosis of the carotid arteries, preferably use less invasive endovascular methods over open surgery. A commonly used endovascular method is angioplasty, which entails the intravascular expansion of a balloon at the site of the stenosis, thereby compressing the occluding plaque. This step is usually followed by the placement of a stent at the site to prevent reclosure of the vessel.

Unfortunately, the use of such devices in the carotid arteries may be accompanied by the risk of dislodging or forming endovascular emboli. These emboli can be rapidly carried into the brain via the internal carotid artery (“ICA”), conducted by the natural antegrade blood flow. Once in the brain, these emboli may become lodged in the small capillaries, potentially causing stroke or other severe consequences to the patient. Accordingly, it would be desirable to provide a device and method that reduces the risk of emboli migrating to the brain prior to, during, and after endovascular procedures.

The following patents and specifications are hereby incorporated by reference: U.S. Pat. Nos. 3,726,269; 4,033,331; 4,169,464; 4,573,966; 4,925,445; 4,935,017; 5,120,323; 5,163,906; 5,199,951; 5,203,776; 5,215,540; 5,219,355; 5,267,982; 5,290,229; 5,304,131; 5,342,306; 5,348,545; 5,368,566; 5,389,090; 5,458,574; 5,462,529; 5,480,380; 5,484,412; European Patent Specifications Nos. 0 339 799 B1 and 0 277 366 A1; and PCT International Patent Application No. WO 96/26758.

### SUMMARY OF THE INVENTION

In view of the foregoing, it is an object of this invention to provide a method and apparatus to prevent cerebral embolization during the angioplasty and stenting of the carotid arteries.

It is another object of this invention to prevent cerebral embolization by establishing temporary retrograde blood flow in the endovascular carotid artery at selected times during the endovascular procedure, thereby redirecting emboli from the internal carotid artery to the external carotid artery.

It is also an object of this invention to block the internal carotid artery distal to the stenosis at appropriate times during the endovascular procedure, thereby reducing the risk that emboli will migrate to the brain, yet allowing continued antegrade blood flow from the common carotid artery to the external carotid artery.

It is also an object of this invention to block both the common and internal carotid arteries at appropriate times during an endovascular procedure, thereby providing a quiescent environment for the angioplasty and stenting procedures, that reduces the risk of emboli migrating into the internal carotid artery.

It is also an object of this invention to allow the operator to selectively control the blood flow in the carotid arteries in coordination with the endovascular procedure to reduce the risk of embolization.

It is also an object of this invention to allow the introduction into the endovascular space devices necessary for the angioplasty and stenting procedures and to position these devices at the site of the stenosis, without obstructing the control of the blood flow in the aforementioned manners.

To accomplish the foregoing objects, the present invention provides a hollow guide catheter and an inflatable guide catheter balloon, which may be introduced into the endovascular space of the common carotid artery (“CCA”), proximal to the carotid bifurcation and the stenosis. Expanding the guide catheter balloon against the vascular wall occludes blood flow in the common carotid artery, thereby inducing retrograde blood flow from the internal carotid artery (“ICA”) to the external carotid artery (“ECA”). The lumen of the guide catheter allows a plurality of wires, balloons, catheters, and stents to be introduced into the endovascular space as may be necessary. The guide catheter also may provide a means to introduce tracers, contrast agents, and other materials into the endovascular space, or a means to aspirate material from the arterial lumen to an extravascular or extracorporeal reservoir.

The present invention further provides a soft-tipped wire with an inflatable balloon attached at or near its distal end. The wire and balloon may be introduced into the arterial endovascular space via the lumen of the aforementioned guide catheter. The wire and distal balloon are independently movable with respect to the guide catheter, and may be positioned at a point distal to the stenosis within the internal carotid artery. The distal balloon may be inflated to occlude antegrade blood flow in the internal carotid artery. The distal balloon and the proximal guide catheter balloon also may be inflated concurrently to reduce the risk that emboli will be transported in the internal carotid artery. Such inflation of both balloons additionally stops blood flow in the vicinity of the stenosis.

Methods of using the apparatus of the present invention are described herein.

### BRIEF DESCRIPTION OF THE DRAWINGS

The above and other objects and advantages of the present invention will be apparent upon consideration of the following detailed description, taken in conjunction with the accompanying drawings, in which like reference numbers reference to like parts throughout, and in which:

FIG. 1 shows initial placement of the guide catheter of the present invention;

FIG. 2 shows the guide catheter balloon inflated;

FIG. 3 shows a soft-tipped wire with a distal balloon being advanced past the stenosis;

FIG. 4 shows the distal balloon inflated;

FIG. 5 shows the guide catheter balloon deflated;

FIG. 6 show both balloons inflated;

FIG. 7 shows the angioplasty being performed;

FIG. 8 shows a stent being delivered into the closed system;

FIG. 9 shows the stent being deployed;

FIG. 10 shows the stent in place;

FIG. 11 show the distal balloon deflated;

FIG. 12 show the distal balloon re-inflated;

FIG. 13 shows the guide catheter balloon deflated;

FIG. 14 shows the guide catheter balloon inflated;

FIG. 15 shows the distal balloon deflated;

FIG. 16 shows the guide catheter balloon deflated;

FIG. 17 is a schematic view of the vascular tree with the guide catheter system of the preferred embodiment of the apparatus of the present invention present therein;

FIG. 18 is a schematic view showing the dilator selecting the origin of a blood vessel;

FIG. 19 is a close-up view similar to FIG. 18;

FIG. 20 is a view similar to FIG. 19, showing the guide wire advanced into a distal blood vessel;

FIG. 21 shows the dilator/guide catheter unit of the preferred embodiment of the apparatus of the present invention in a position in which the inner dilator has reached its intended location;

FIG. 22 shows the dilator/guide catheter unit of the preferred embodiment of the apparatus of the present invention where the guide catheter has been advanced over the inner dilator catheter to the intended location; and

FIG. 23 is a view similar to FIG. 22, after the inner dilator and guide wire have been removed from the guide catheter.

### DETAILED DESCRIPTION OF THE INVENTION

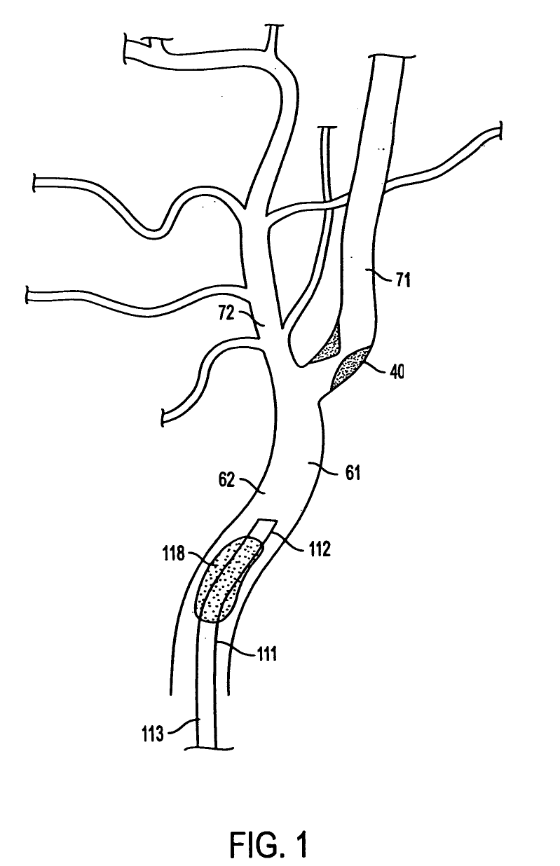

Referring to FIG. 1, the apparatus of the invention comprises a guide catheter system comprising a hollow guide catheter (111). The guide catheter further comprises a stiff proximal shaft (113), and a soft atraumatic tip (112). These properties allow the catheter to be controllably navigated within an intravascular space, such as the lumen of the common carotid artery (62), while minimizing the risk of injury to the vessel walls. A working lumen of the catheter provides a conduit for multiple apparatus to be independently introduced or withdrawn into the intravascular lumen. The catheter lumen also provides a conduit for fluid communication between the lumen and the exterior of the body. This conduit may allow reagents or other substances to be introduced into or aspired from the lumen interior.

The apparatus further comprises a guide catheter balloon (118) disposed near the distal end (112) of the guide catheter (111). This balloon may be integral with the catheter, or formed separately and attached to the catheter. The guide catheter balloon (118) may be selectively and controllably inflated, thereby expanding against the walls of the surrounding vessel. For the duration of this expansion the vessel is occluded and the distal blood flow is stopped. The risk of upstream passage of debris, such as emboli, within the ICA is accordingly reduced.

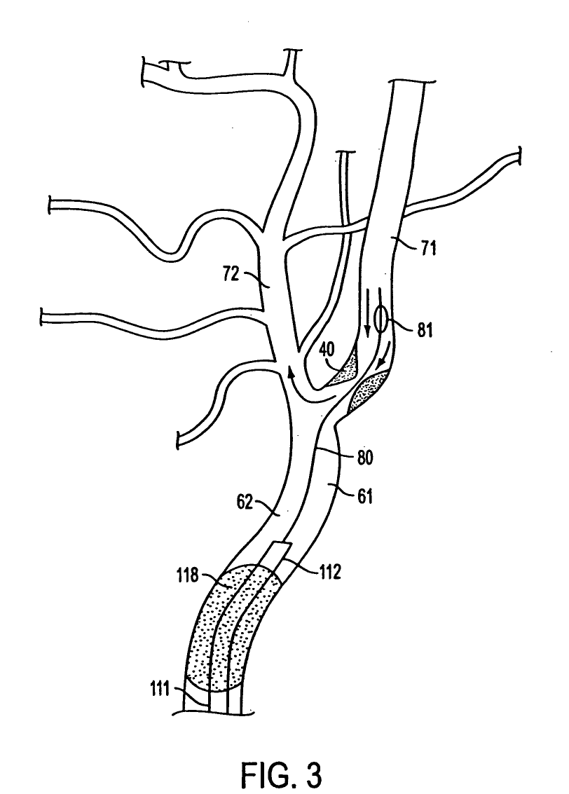

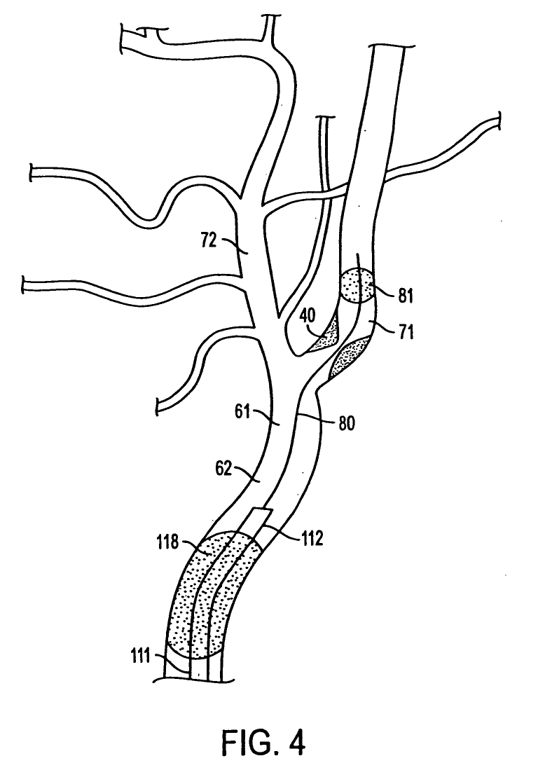

Referring to FIGS. 3 and 4, the apparatus further comprises a soft-tipped distal wire (80). This wire may be introduced into the intravascular lumen via the conduit provided by the guide catheter (111). The distal wire (80) is approximately coaxial with the guide catheter (111), and may extended or retracted independently of the catheter, thereby allowing the wire to be retracted within the catheter or to be extended beyond the distal end of the catheter.

The apparatus further comprises a distal balloon (81) associated with and disposed near the distal end of the wire (80). The distal balloon (81) is selectively and controllably inflatable, and may be inflated or deflated independently of the guide catheter balloon (118). In a manner analogous to the guide catheter balloon (118), intravascular inflation of the distal balloon (81) will occlude the surrounding vessel, preventing the passage of blood or debris.

The apparatus further comprises medical and surgical devices such as angioplasty balloons (91), angioplasty catheters (90) and stents (50). These devices may be introduced into the intravascular lumen via the hollow guide catheter (111), and therein may be moved and actuated independently of the guide catheter (111), the distal wire (80), or their associated balloons (118) or (81).

A method of using the apparatus of the invention is now described. As shown in FIG. 1 and described hereinbelow, the invention is preferably deployed and actuated to treat a stenosis in the internal carotid artery (ICA). However, it is understood that the apparatus and method may be used in a similar manner in other locations within the circulatory system where appropriate.

In FIG. 1, the guide catheter (111) and associated guide catheter balloon (118) may be positioned in the lumen of the common carotid artery (CCA) (61) by methods known in the art. For example, a diagnostic cerebral catheter can be used to evaluate the path to the site of stenosis, which is then exchanged for a guide catheter by using a safe “neuro” exchange wire.

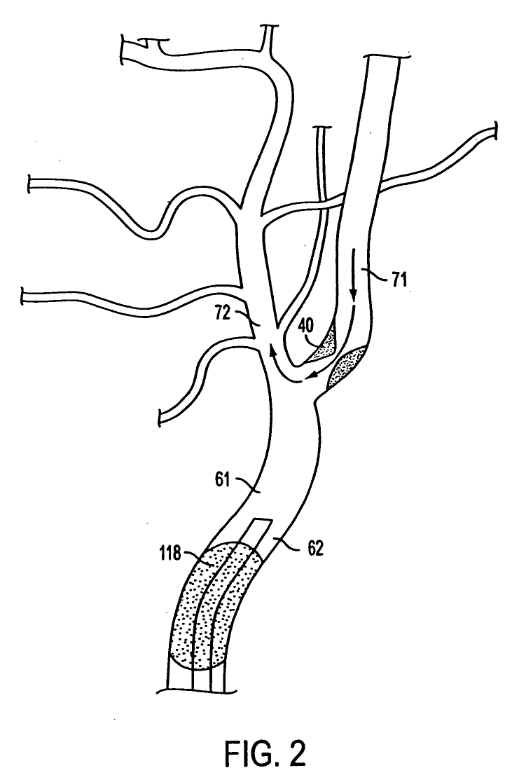

Referring to FIGS. 1 and 2, the operator introduces the guide catheter (111) and deflated balloon (118) into the CCA (61). The guide catheter balloon is positioned at a location (62) proximal to and hence upstream of the bifurcation of the CCA (61). The balloon (118) is inflated and expanded against the wall of the CCA (61), occluding the vessel. Without the antegrade flow from the CCA, a retrograde blood flow is created distal to the guide catheter balloon (118). This retrograde blood flow travels from the high-pressure ICA (71) to the lower pressure external carotid artery (ECA) (72). Any debris formed during the subsequent procedures are thereby prevented from being carried into the brain via the ICA (71) and instead are safely conducted to the ECA (72).

In FIG. 3, a soft-tipped distal wire (80) with an inflatable balloon (81) attached to its distal end is introduced into the ICA (61) via the lumen of the guide catheter (111). The wire (80) and its balloon (81) are advanced beyond the end of the guide catheter (111) and past the stenosis (40) in the ICA. Due to the retrograde flow created by the occlusion of the CCA (61), emboli that may be dislodged or formed during this passage will be conducted safely from the ICA (71) and into the ECA (72). Prior to inflating the distal balloon (81), the stenosis site may be aspirated with the guide catheter to remove debris or other emboli. In FIG. 4, the distal balloon (81) is inflated to occlude the ICA (71) distal to the stenosis (40). This occlusion stops all blood flow in the vicinity of the stenosis (40), and physically blocks the ICA from conducting debris into the cerebrum of the brain.

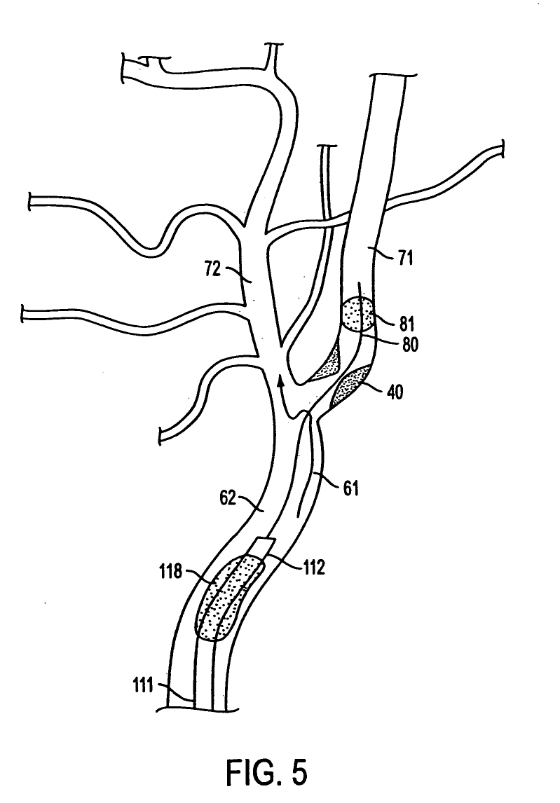

In FIG. 5, the guide catheter balloon (118) is deflated, restoring antegrade blood flow from the CCA (61), which clears out the area surrounding the stenosis (40) into the ECA (72). No material is allowed to proceed down the ICA (71) due to the blockage conferred by the distal balloon (81).

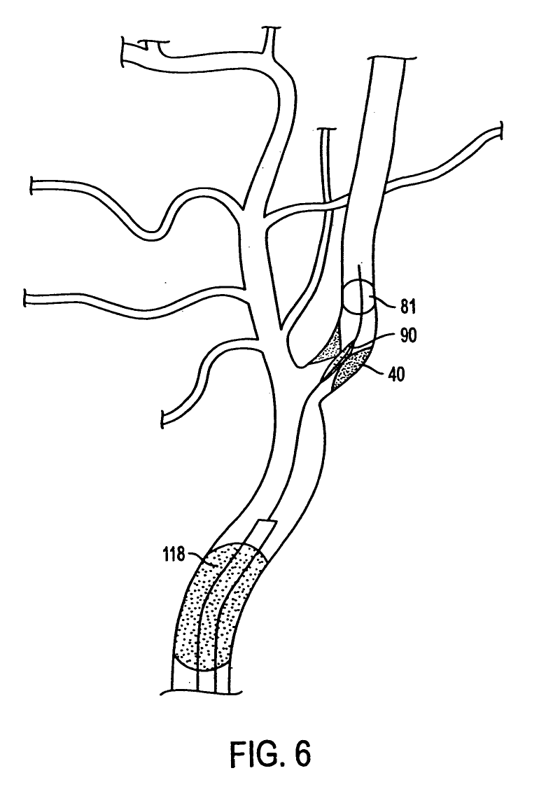

In FIG. 6, the guide catheter balloon (118) is re-inflated, occluding antegrade blood flow from the CCA (61) and hence stopping all blood flow in the vicinity of the stenosis (40). The inflation of both balloons in this manner also provides a quiescent environment around the stenosis for the subsequent procedure. The angioplasty catheter (90) and balloon (91) are introduced into the carotid endovascular space via the lumen of the guide catheter (111). Both the angioplasty and stent use devices and methods known to one skilled in the art, and will not be described herein. Once introduced into the vessel lumen, the angioplasty devices are navigated to the location of the stenosis (40).

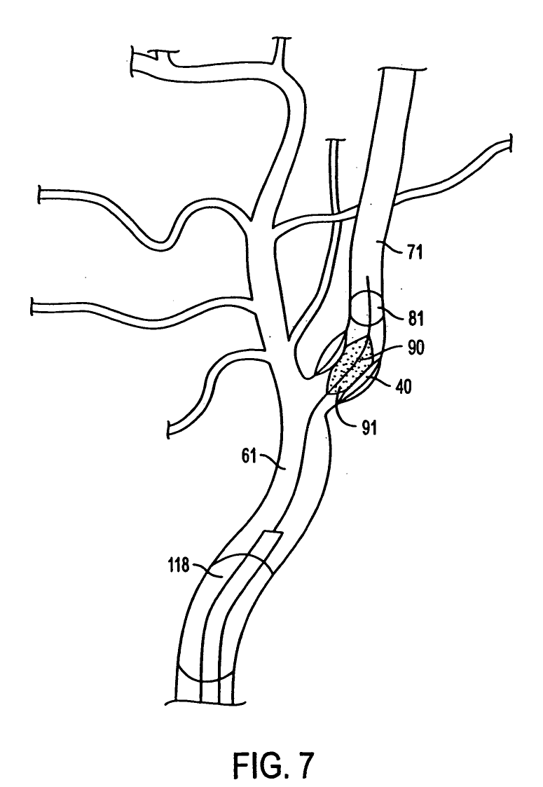

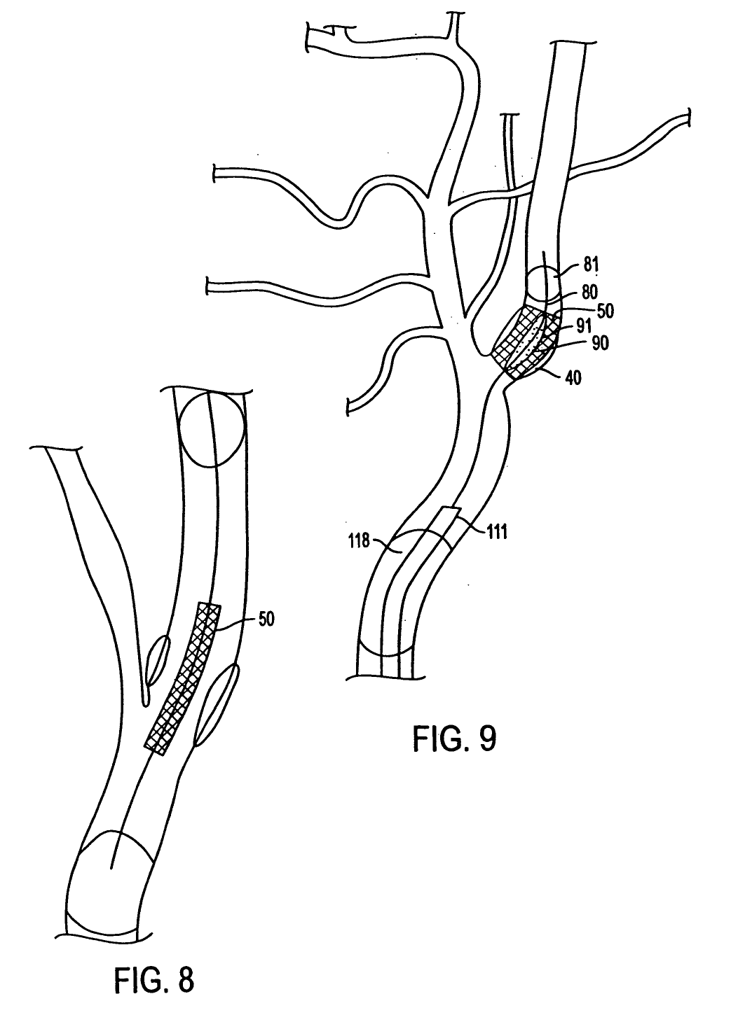

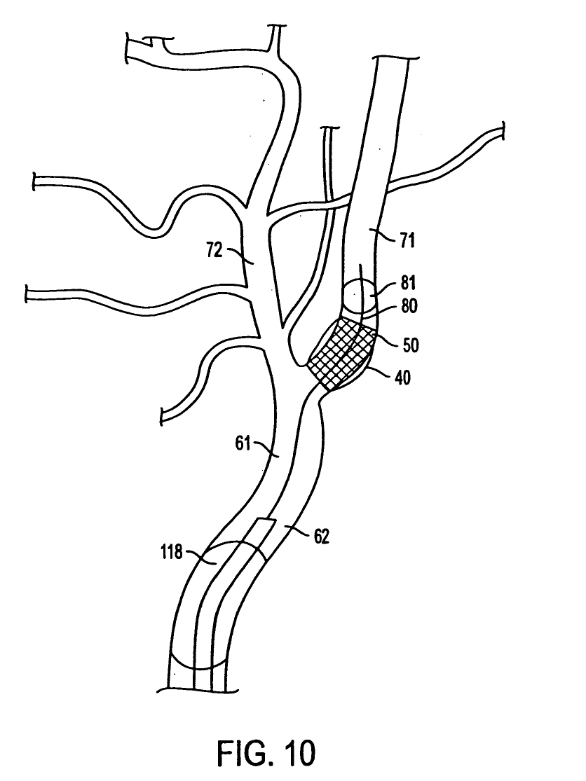

In FIG. 7, the angioplasty is performed at the location of the stenosis (40), thereby widening the ICA lumen by compressing the plaques of the stenosis. In FIGS. 8 and 9, the stent (50) is delivered into the ICA via the guide catheter (111) and subsequently installed at the repaired stenosis.

In FIG. 10, the angioplasty catheter (90) and balloon (91) are withdrawn back through the guide catheter (111), leaving the stent in place (40). Since both balloons remain inflated throughout the angioplasty and stent procedures, the cerebrum is continuously protected from any emboli created or dislodged during the procedure.

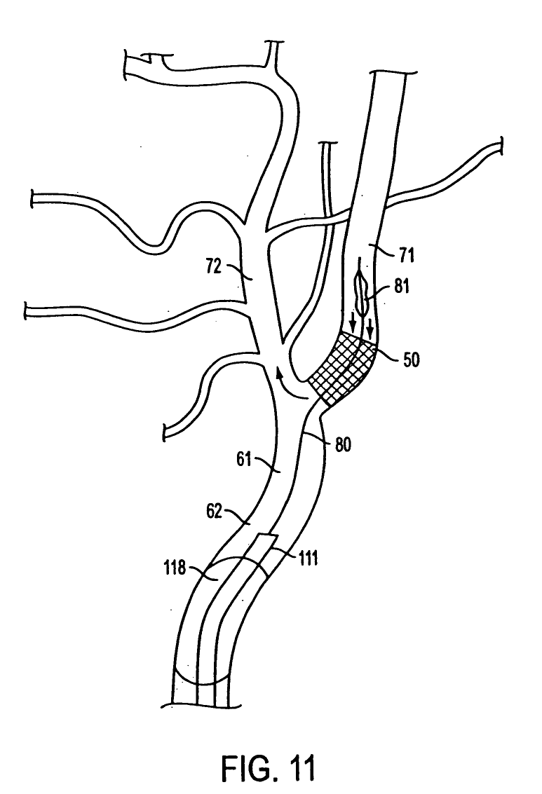

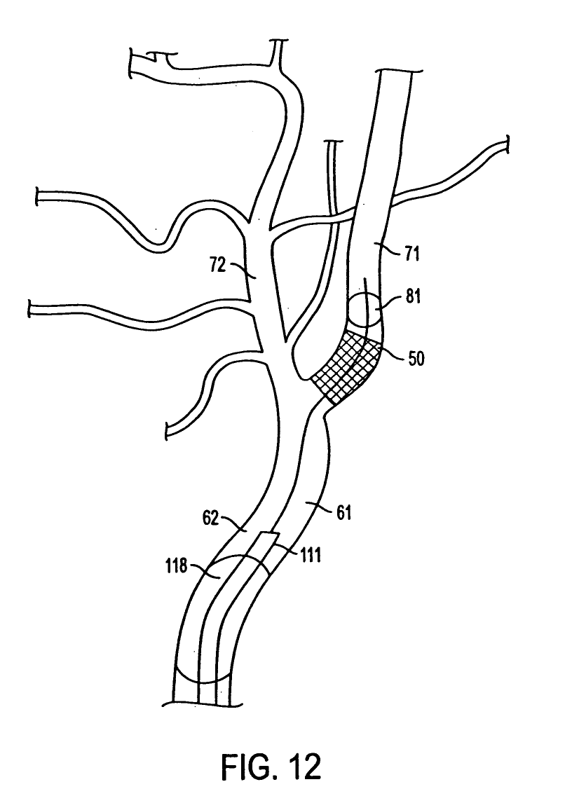

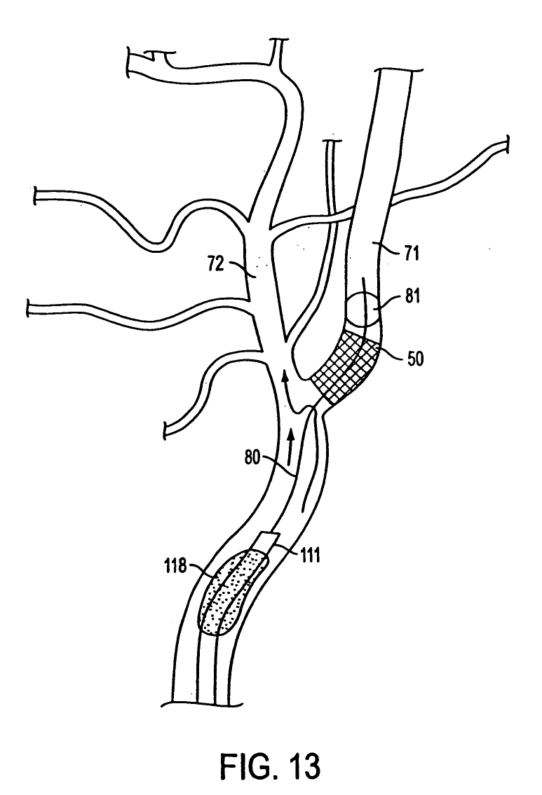

In FIG. 11, the distal balloon (81) is deflated, restoring the retrograde blood flow from the ICA (71) into the ECA (72). This retrograde flow clears any emboli formed during the procedure into the ECA (72). In FIG. 12, the distal balloon (81) is then re-inflated, again stopping flow in the internal carotid artery (71).

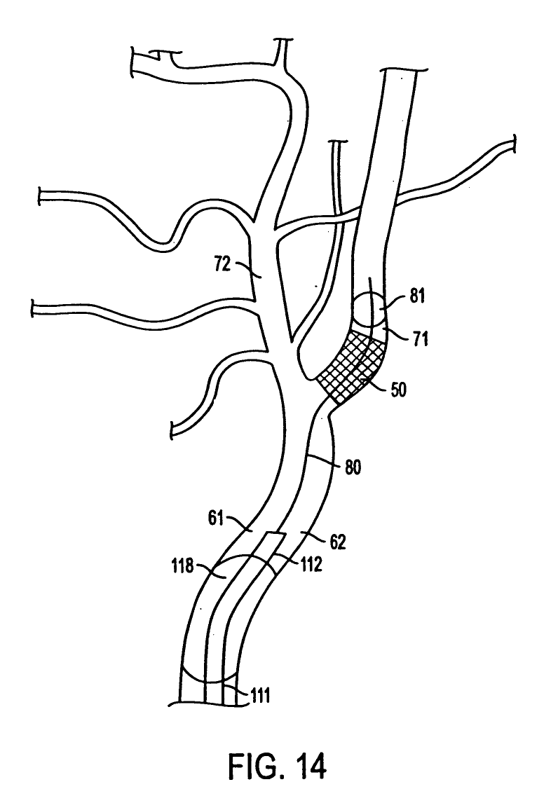

In FIG. 13, the guide catheter balloon (118) is deflated, restoring antegrade flow from the CCA (61) to the ECA (72). This flow also has the effect of clearing any remaining emboli from the repaired stenotic region into the ECA (72). Once completed, the guide catheter balloon (118) is re-inflated as shown in FIG. 14.

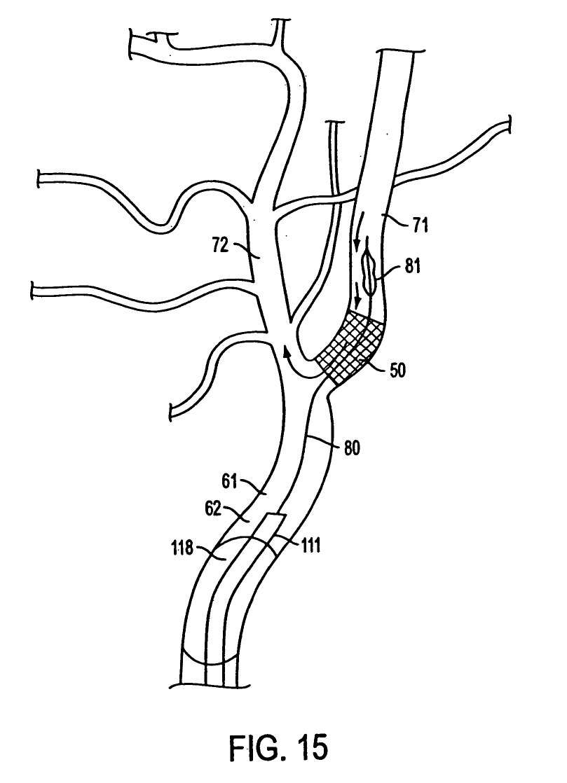

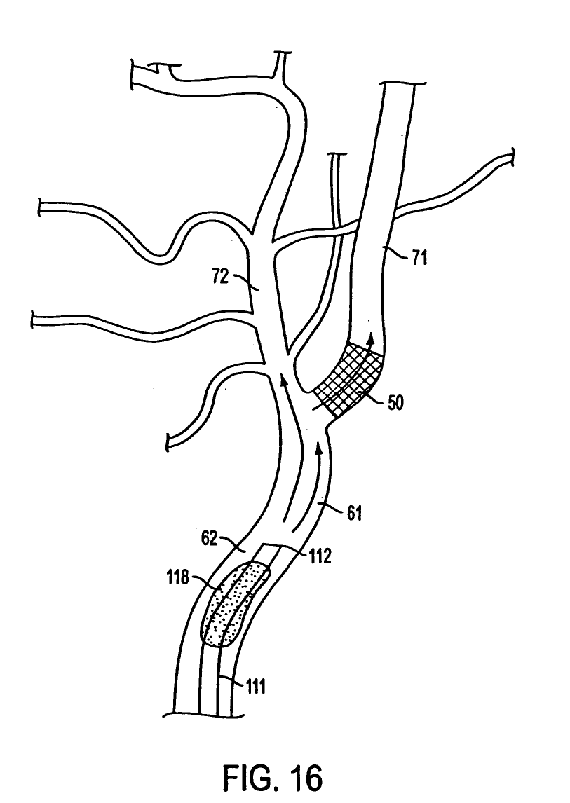

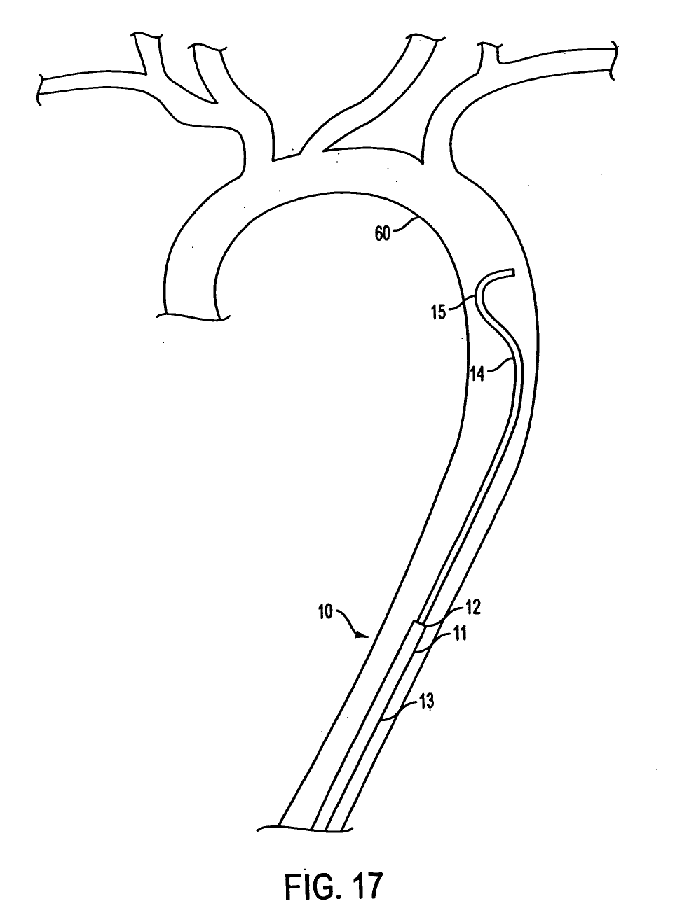

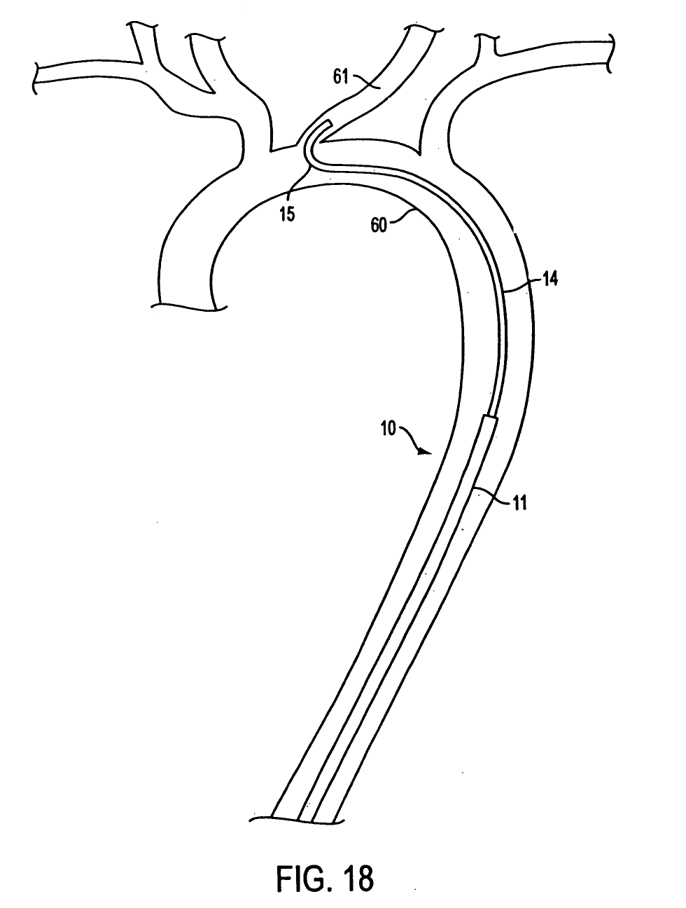

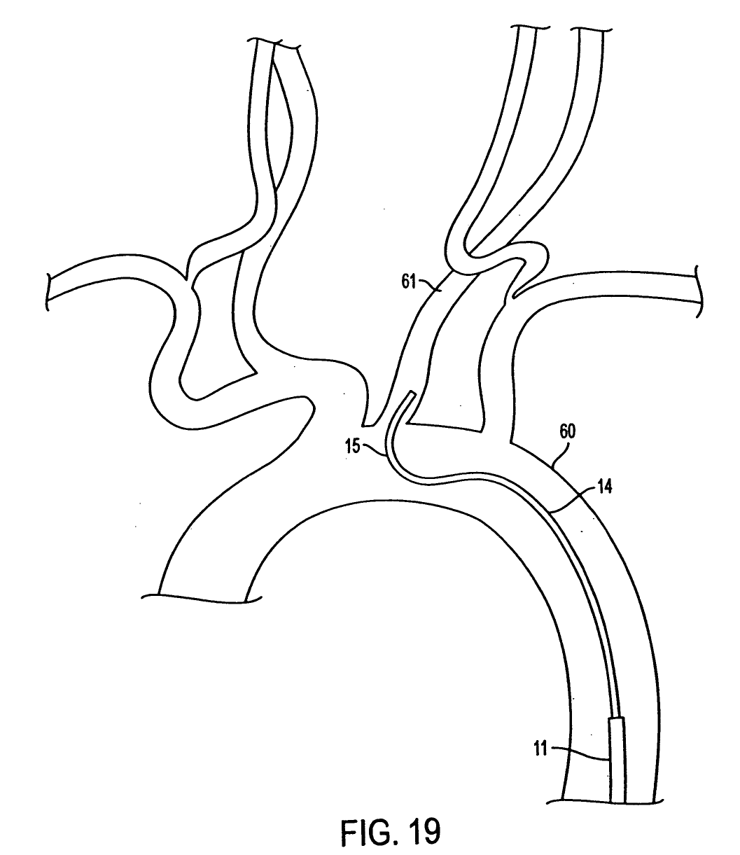

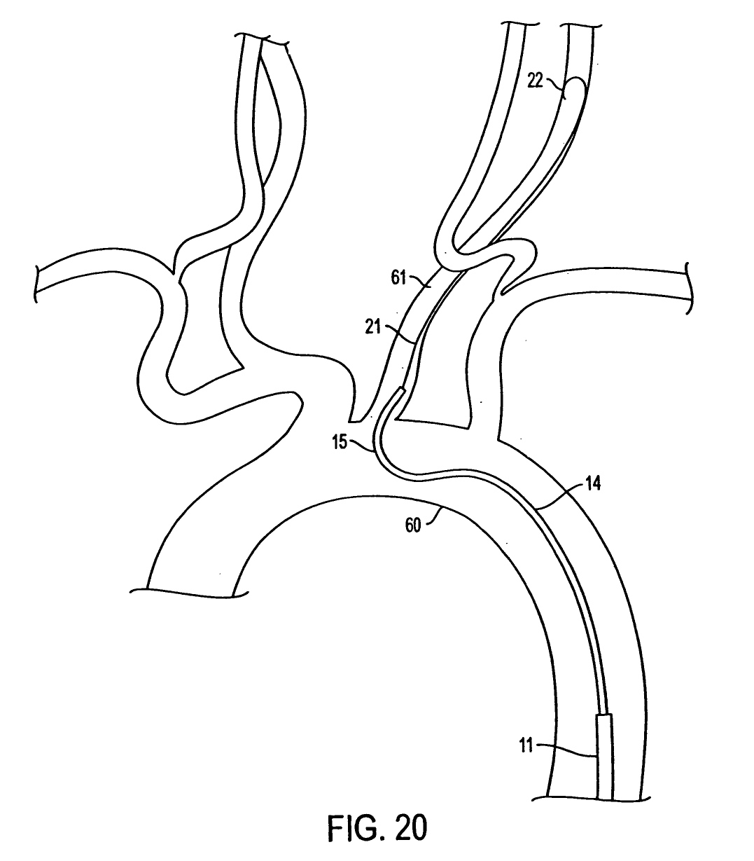

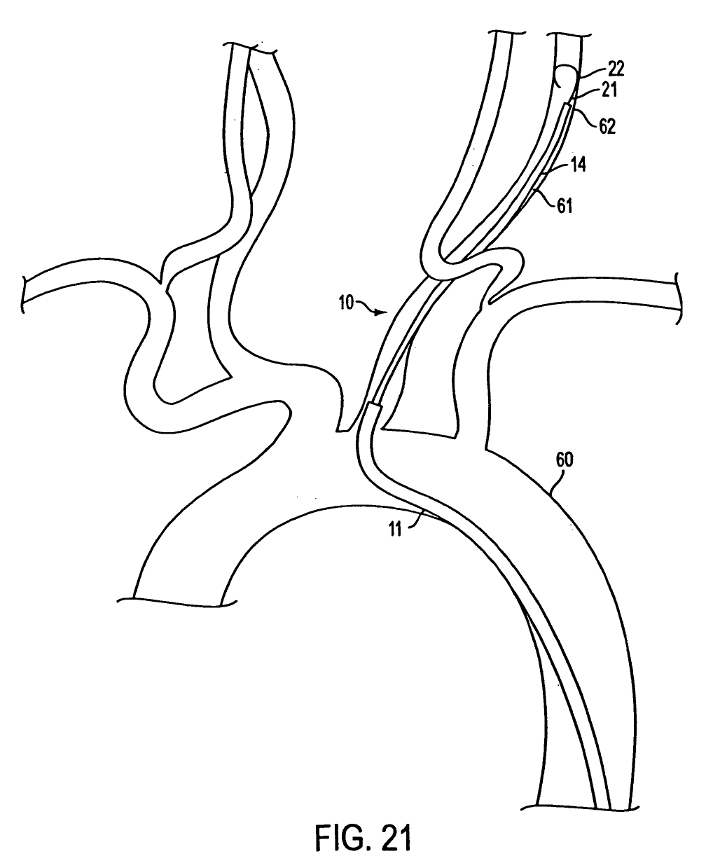

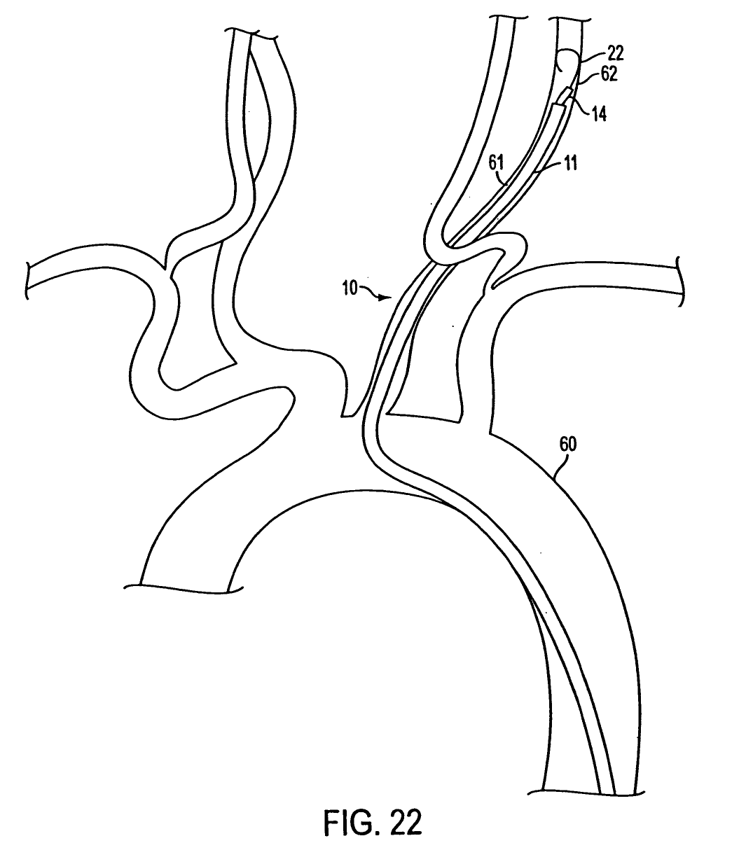

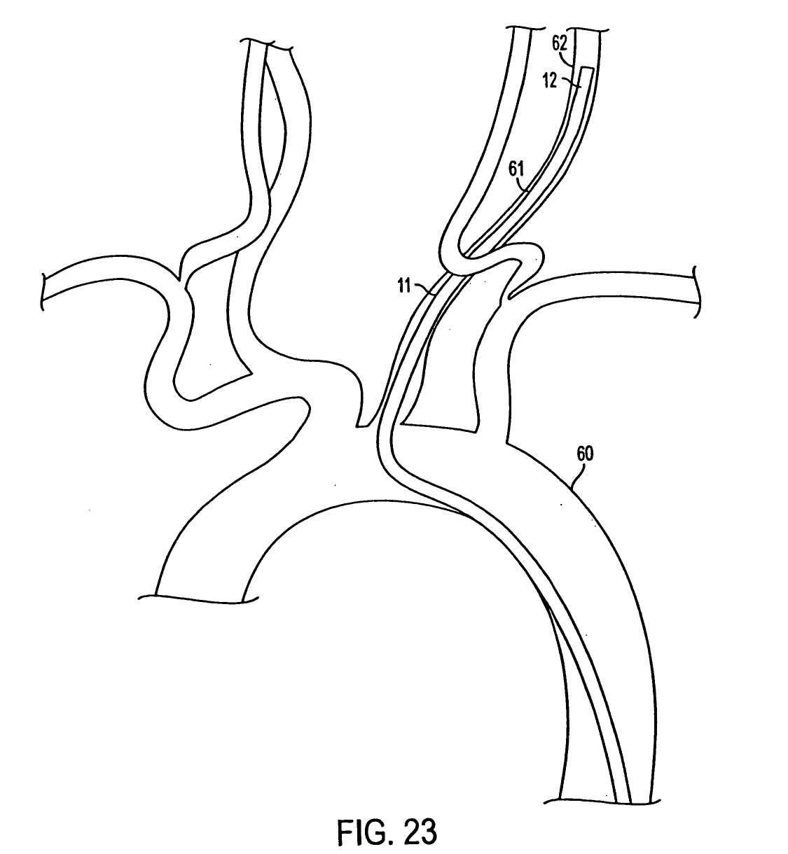

In FIG. 15, the distal balloon (81) is deflated, restoring retrograde blood flow in the area of the corrected stenosis. The distal wire (80) and deflated distal balloon (81) are withdrawn past the repaired stenosis and removed from the endovascular space. Any emboli created in the ICA (71) during this extrication will be drawn into the ECA (72) as a consequence of the retrograde flow. In FIG. 16, once the wire and distal balloon are withdrawn, the guide catheter balloon (118) is deflated, restoring normal antegrade blood flow to the CCA (61), the ICA (71) and the ECA (72). The guide catheter (111) and balloon (118) can be removed from the patient.

The lumen of guide catheter (111) may communicate with the extracorporeal space under control of the operator. The operator may use this feature of the guide catheter to remove or introduce reagents or devices into the endovascular space of the carotid arteries. These reagents include, but are not limited to, contrast media or tracing agents. The introduction of these reagents at appropriate times during or after the procedure allows the operator to visualize and to evaluate the progress and integrity of the stenosis and its repair.

It is understood that the embodiments described are not meant as limitations to either the preferred device or method, since further modifications or variations to the invention would be apparent to one skilled in the art. Such modifications or variations could be introduced without departing from the principles of the present invention and would be within the scope of the claims.

1-13. (canceled)

14. Apparatus for performing a therapeutic procedure on a bifurcated artery having a first branch vessel and a second branch vessel comprising:
a catheter having a blood inlet port at the distal end, a blood outlet port at the proximal end, a lumen extending between the blood inlet port and the blood outlet port;
an occlusion element disposed on the distal end of the catheter, the occlusion element having a contracted state suitable for transluminal insertion and an expanded state wherein the occlusion element is configured to occlude antegrade flow in the artery proximal to the bifurcation;
a syringe adapted for connection to the blood outlet port of the catheter to provide intermittent aspiration through the lumen of the catheter; and
an emboli-blocking element configured to occlude flow in a branch vessel of the bifurcation.
